(19) United States
(12) Patent Application Publication
(10) Pub. No.: US 2004/0254144 A1
(43) Pub. Date: Dec. 16, 2004

(54) Use of inhibitors of heparin-binding epidermal growth factor or inhibitors of its receptors for the preparation of drugs useful for treating myeloma

(76) Inventors: Bernard Klein, Saint Clement De Riviere (FR); John De Vos, Saint Mathieu De Treviers (FR); Yue Dan Wang, Suzhou Po (CN)

Correspondence Address: DENNISON, SCHULTZ, DOUGHERTY & MACDONALD, 1727 KING STREET, SUITE 105, ALEXANDRIA, VA, 22314, US

(21) Appl. No.: 10/484,819

(22) PCT Filed: Aug. 1, 2002
(86) PCT No.: PCT/FR02/02777

(30) Foreign Application Priority Data
Aug. 1, 2001 (FR) 01/10354

(51) Int. Cl. A61K 31/727; A61K 39/395

(52) U.S. Cl. 514/56; 424/145.1

(57) ABSTRACT

The present invention relates to the use of at least one inhibitor of heparin-binding (HB) epidermal growth factor (EGF), or at least one inhibitor of HB-EGF receptors, or ErbB receptors, or at least one inhibitor of associated transduction pathways for the preparation of drugs useful for inducing the apoptosis and/or inhibiting the proliferation of IL-6-dependent plasmocytic tumor cells.

The present invention relates to the treatment of multiple myeloma. It relates more particularly to the use of at least one inhibitor of heparin-binding (HB) epidermal growth factor (EGF), or at least one inhibitor of HB-EGF receptors, or ErbB receptors, or at least one inhibitor of associated transduction pathways for the preparation of drugs useful for inducing apoptosis and/or inhibiting the proliferation of IL-6-dependent plasmocytic tumor cells.

The present invention further relates to the use of at least one inhibitor of heparin-binding epidermal growth factor, HB-EGF, or at least one inhibitor of HB-EGF receptors, or ErbB receptors, or at least one inhibitor of associated transduction pathways, in combination with at least one IL-6 inhibitor, or at least one IL-6 receptor inhibitor, or at least one inhibitor of associated transduction pathways, for the preparation of drugs useful for inducing apoptosis and/or inhibiting the proliferation of IL-6-dependent plasmocytic tumor cells.

Interleukin-6 (IL-6) and the other cytokines of the IL-6 family are important growth factors of plasmocytic malignant cells involved in multiple myeloma^{(1,2)}.

It is also known that IL-6 is principally produced by cells in the bone marrow environment (2,3) and that the production of IL-6 by these cells is induced after interaction with myeloma cells^{(4,5)}.

It has now been found that the gene coding for heparin-binding epidermal growth factor (HB-EGF) is overexpressed in myeloma cells and that the IL-6-induced proliferation of myeloma cell lines is linked to the presence of a CD9/HB-EGF/ErbB1 autocrine loop.

HB-EGF is a factor produced either in soluble form or in the form of a transmembrane protein^{(6,7)}. The membrane form is the diphtheria toxin receptor. Also, HB-EGF is a ligand of the epidermal growth factor receptors (ErbB1 and ErbB4)^{(6,7)}. It is produced by various tumor cells and acts as an autocrine tumoral growth factor^{(6,7)}.

The HB-EGF inhibitors which are suitable for the purposes of the invention are any substances capable of inhibiting the proliferation or inducing the apoptosis of plasmocytic tumor cells, for example under the conditions defined in the illustrative Examples below.

Examples which may be mentioned in particular of substances capable of inhibiting HB-EGF are heparins, especially low molecular heparin, diphtheria toxin and anti-HB-EGF antibodies, especially anti-HB-EGF monoclonal antibodies such as those described in the illustrative Examples below.

The HB-EGF receptor inhibitors which are suitable for the purposes of the invention are any substances capable of inhibiting the proliferation or inducing the apoptosis of plasmocytic tumor cells, for example under the conditions defined in the Examples below.

Examples of appropriate ErbB receptor inhibitors are especially anti-ErbB1 monoclonal antibodies, for example the monoclonal antibody LA-1 marketed by UBI (Lake Placid, N.Y., USA).

Examples of IL-6 inhibitors which can be used for the purposes of the invention are corticoids, mutated IL-6 or other IL-6 inhibitors, anti-IL-6 monoclonal antibodies such as, in particular, those directed against the gp80 chain or gp130 chain, for example the monoclonal antibodies B-E8 produced by Diaclone (Besançon), and IL-6 receptor inhibitors such as the monoclonal antibody B-R3, an anti-IL-6 gp130 transducer antibody, which is the property of INSERM and Diaclone and is produced by Diaclone.

An effective dose of each of the inhibitors employed according to the invention must be used as a pharmacologically equivalent dose deduced from the experimental data.

Of course, the effective dose depends on the state of development of the myeloma, the patient's age, biological profile and clinical condition, and other pharmacological parameters dependent on the patient or his clinical condition, for example the daily production of IL-6 calculated according to the method described by Lu et al.⁽¹³⁾, the proliferation profile, the level of CRP/IL-6, the isotype of the monoclonal protein, the prognostic factors of the myeloma, and the vital functions, especially the creatinine clearance, the hepatic functions, etc.

The effective dose can be determined according to the method described by Lu et al.⁽¹³⁾.

In general, the dose of HB-EGF inhibitor or HB-EGF receptor inhibitor can be between 10 and 1000 μg/ml of plasma.

The dose of IL-6 inhibitor or IL-6 receptor inhibitor can be between 10 and 1000 μg/ml of plasma.

According to another feature, the present invention relates to a pharmaceutical composition with an anti-myeloma action (an inhibitory action on myeloma proliferation) which contains, as the active principle, an effective amount of at least one HB-EGF inhibitor or at least one HB-EGF receptor inhibitor, in combination with a pharmaceutically acceptable excipient.

In one preferred variant, the pharmaceutical composition according to the invention contains, as the active principle, an effective amount of at least one HB-EGF inhibitor or at least one inhibitor of the HB-EGF ErbB receptors, particularly the ErbB1 receptor or the ErbB4 receptor, or at least one inhibitor of transduction pathways, in combination with an effective amount of at least one IL-6 inhibitor, or at least one L-6 receptor inhibitor, or an inhibitor of IL-6-induced transduction pathways, said inhibitors being packaged together or separately with a pharmaceutically acceptable vehicle.

It is possible to use any conventional pharmaceutically acceptable vehicle, for example a solution containing a monoclonal antibody stabilizer or human albumin, it being preferable to use a pharmaceutically acceptable vehicle that is appropriate for parenteral administration.

The invention further relates to a method of treating myeloma which consists in administering to myeloma patients an effective amount of at least one HB-EGF inhibitor, or at least one HB-EGF receptor inhibitor, or at least one inhibitor of associated transduction pathways, optionally in combination with an effective amount of at least one IL-6 inhibitor, or at least one IL-6 receptor, or at least one inhibitor of associated transduction pathways, the administration of said inhibitors being concomitant or sequential and being determined according to data deduced from pharmacological parameters or from clinical data.

The present invention will now be described in greater detail by means of the tests carried out, which demonstrate that, in the case of myeloma, it is possible to inhibit the proliferation of plasmocytic malignant cells or cause the apoptosis of these cells.

The tests reported below were carried out using the human myeloma cell lines (HMCLs) XG-1, XG-6, XG-13 and XG-14 obtained in the Cell Therapy Unit of the Montpellier Teaching Hospital and INSERM Unit U475 in Montpellier, which have been described in the literature^{(8,9,10)}.

It is known that the growth of these four myeloma cell lines, XG-1, XG-6, XG-13 and XG-14, is strictly dependent on the addition of exogenous IL-6. When IL-6 is withdrawn, these cells undergo a progressive apoptosis in 3 to 4 days. The HMCLs were maintained in X-VIVO 20 serum-free culture medium (Biowittaker, Md., US) and 5 ng/ml of IL-6.

The following were used in these tests:

the EGFs and recombinant EGFs marketed by R & D System (Minneapolis, Minn., USA),

the mutated diphtheria toxin marketed by Sigma (St Louis, Mo., USA),

the neutralizing anti-HB-EGF antibody marketed by R & D System,

the neutralizing anti-ErbB1 receptor monoclonal antibody (mAb) LA-1 produced by UBI (Lake Placid, N.Y., USA) and marketed by EUROMEDEX (Souffelweyersheim, France),

the purified goat immunoglobulins marketed by TEBU (Le Perray en Yvelines, France), and

the neutralizing anti-IL-6 gp130 transducer monoclonal antibody B-R3 described by Wijdenes et al.⁽¹¹⁾.

The methods used in these tests will now be described in detail.

Expression of Intercellular Signal Genes in Myeloma Cells

The expression of 268 genes coding for intercellular signal proteins was evaluated on myeloma cell lines (HMCLs) and lymphoblastoid cell lines (LCLs) infected with Epstein-Barr virus (LCL) using ATLAS DNA membranes according to the Clontech technique (Basle, Switzerland).

The poly (A+) RNA was extracted from each cell and used to synthesize cDNA labeled with a radioactive element (³²P).

The radiolabeled cDNAs were then hybridized with two identical DNA chips according to the technique recommended by Clontech, and the radioactivity was analyzed by Phospho Imager (Amersham, Saclay, France).

Analysis by Flux Cytometry

The expression of ErbB1 was evaluated by incubating 5×10⁵ myeloma cells with 0.5 μg of a mouse monoclonal antibody directed against human EGF receptor (anti-EGF-R) (LA-1) or a mouse monoclonal antibody that does not recognize human antigens (Immunotech, Marseille, France), in phosphate buffer (PBS) containing 30% of AB serum, at 4° C. for 30 minutes. The cells were then washed and incubated with an anti-mouse goat monoclonal antibody conjugated with polyethylene glycol (PE) (Immunotech, Marseille, France), in PBS containing 30% of AB serum, at 4° C. for 30 minutes.

The membrane HB-EGF was detected by labeling 5×10⁵ myeloma cells with 0.5 g of anti-human HB-EGF goat antibodies or 1% of goat serum in PBS containing 100 μg/ml of immunoglobulins (Ig), at 4° C. for 30 minutes. The cells were washed and incubated with anti-goat pig immunoglobulins conjugated with FITC, in PBS containing 100 μg/ml, at 4° C. for 30 minutes. The percentage of labeled cells and the mean fluorescence intensity (MFI) were determined with a FACScan flux cytometer (Becton Dickinson, USA) or some other type of flux cytometer.

Cell Proliferation Tests

The cells were cultivated for 5 days in 96-well flat-bottom microtiter plates at a rate of 10⁴ cells/well in X-VIVO 20 serum-free culture medium. Different concentrations of cytokines, growth factors or cytokine/growth factor inhibitors were added to 6 culture wells per group at the start of the culture. At the end of the culture, the cells were labeled with tritiated thymidine (Amersham, Orsay, France) for 12 hours, harvested and counted by the procedure described by De Vos et al.⁽¹²⁾.

Long-Term Growth of Myeloma Cells

To examine the effects of EGF or IL-6 on the long-term growth of myeloma cells, the cells were washed once with culture medium, incubated for 5 h at 37° C. in X-VIVO 20 culture medium and washed a further twice.

They were then cultivated at a cell concentration of 10⁵ cells/ml with HB-EGF (50 μg/ml) or IL-6 (500 μg/ml), with or without 10 μg/ml of neutralizing anti-gp130 monoclonal antibody B-R3 (INSERM/Diaclone) or with or without 10 μg/ml of neutralizing anti-ErbB1 monoclonal antibody (LA-1).

Detection of Apoptotic Cells

The myeloma cells were cultivated for 3 to 4 days in flat-bottom microplates at a rate of 3×10⁵ cells per well in X-VIVO 20 culture medium with different amounts of IL-6/HB-EGF or IL-6/HB-EGF inhibitors.

At the end of the culture, the cells were washed twice with PBS and suspended in a solution of annexin V-FITC (1/50 dilution in HEPES buffer: 10 mM HEPES/NaOH, pH 7.4, 140 mM NaCl and 5 mM CaCl₂).

They were incubated for 20 minutes at room temperature and washed twice with HEPES buffer. The fluorescence was analyzed with a FACScan flux cytometer. cDNA was produced with a total of 2 μg of RNA using the reverse transcriptase Superscript II (Life Technologies) and oligo d(T)_{12-1R} (Amersham Pharmacia Biotech) as primer. Each 25 μl portion of PCR contained 1 μl of cDNA leading strand, 1 μM of each primer (sense and antisense), 0.2 mM of each dNTP, 1.5 mM MgCl₂, 1× buffer for polymerase, 2 U of Taq polymerase (Life Technologies) and 1 μCi of α-³²P-dCTP (Amersham Pharmacia Biotech). The following primers were used:

Tyro3
5′-CAC TGA GCT GGC TGA CTA AGC CCC (sense) and
5′-AAT GCA TGC ACT TAA GCA GCA GGG (antisense);
HB-EGF
5′-TGG TGC TGA AGC TCT TTC TGG (sense) and
5′-GTG GGA ATT AGT CAT GCC CAA (antisense);
FRZB
5′-AAG TCT GGC AGG AAC TCG AA (sense) and
5′-ACT TCC TGG TGC TTG ATT GC (antisense);
β₂-microglobulin (β₂-M)
5′-CCA GCA GAG AAT GGA AAG TC (sense) and
5′-GAT GCT GCT TAC ATG TCT CG (antisense).

The sizes of the PCR products were as follows: Tyro3=344 pdb, HB-EGF=605 pdb, FRZB (Frizzled-related receptor B)=599 pdb, β₂-M=269 pdb. The amplification profile was 1 minute at 94° C., 45 seconds at 59° C. (Tyro3) or 62° C. (HB-EGF) or 60° C. (FRZB or β₂-M) and 1 minute at 72° C., these operations being followed by a final extension of 10 minutes at 72° C. The number of cycles was 26 for Tyro3, 32 for HB-EGF and 25 for FRZB or β₂-M. The reaction products were subjected to electrophoresis on 4% polyacrylamide gel, dried and exposed to X-ray films.

### EXAMPLE 1

### Critical Action of Autocrine HB-EGF on the Survival and Proliferation of Myeloma Cells

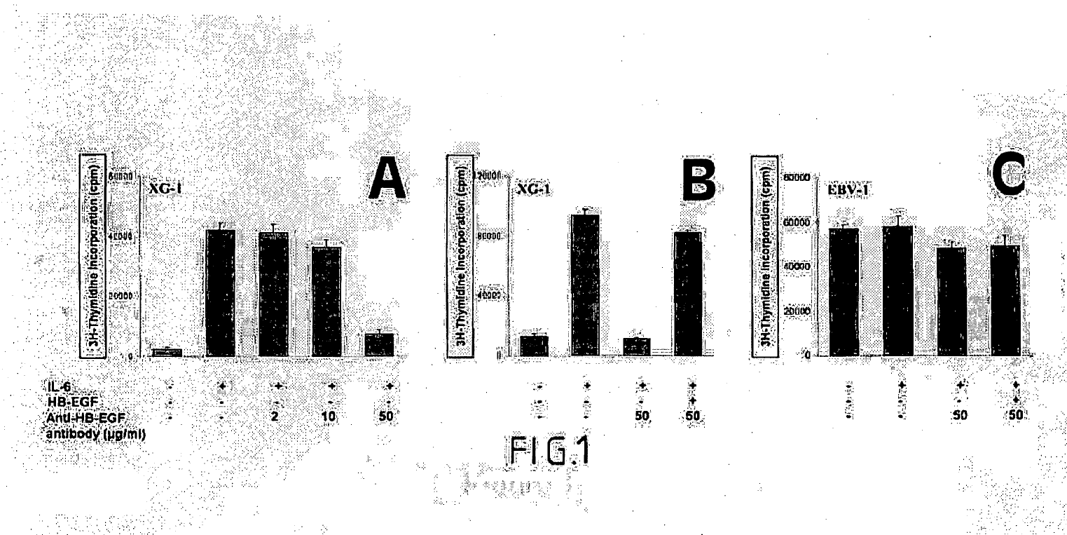

HB-EGF is a gene whose expression can be linked to the pathobiology of multiple myeloma (MM). By using DNA chips, it was found that the HB-EGF gene was markedly overexpressed in 3 myeloma lines (HMCLs XG-1, XG-7 and XG-14) but in none of the 4 LCLs. The expression of the HB-EGF gene was investigated by RT-PCR in cell lines and primary cells. The mRNA of HB-EGF was detected in 3/6 HMCLs, but in none of the 4 LCLs, which confirms the results obtained with the DNA chips. Interestingly, whereas the mRNA of HB-EGF could not be amplified by RT-PCR in purified malignant plasma cells from 4 out of 4 cases of PCL, a strong expression was found in purified bone marrow cells from 2 patients suffering from MM. In normal plasma cells, a weak expression was noted in 1 out of 4 samples. In contrast to the ErbB4 gene, the ErbB1 gene was highly expressed in the MM cells and in the LCLs, which suggests that HB-EGF may be an autocrine growth factor of tumor cells by binding to its ErbB1 receptor. An investigation was therefore made to see whether blocking of the HB-EGF activity could modulate the proliferation of the XG-1 MM cell line, which highly expressed the HB-EGF gene. As emphasized in FIG. 1A, the addition of a neutralizing antibody to HB-EGF blocked the proliferation of XG-1 in a dose-dependent manner. With 50 μg/ml of anti-HB-EGF antibody, the inhibition rose to 80%. This inhibitory effect was reversed by the addition of excess recombinant HB-EGF, which demonstrates the specificity of the antibody blocking effects (FIG. 1B). By contrast, the anti-HB-EGF antibody had no effect on the proliferation of EBV-1 LCL (FIG. 1C).

These observations clearly show that HB-EGF is a novel growth factor involved in the survival of IL-6 and the proliferation of XG-1 myeloma cells.

Membrane HB-EGF was also identified on myeloma cells by incubating these incubated cells with anti-HB-EGF goat antibodies or control goat serum and then with an anti-goat Ig pig antibody conjugated with FITC. The fluorescence was analyzed with a FACScan cytofluorimeter. The results are those of one experiment representative of two experiments.

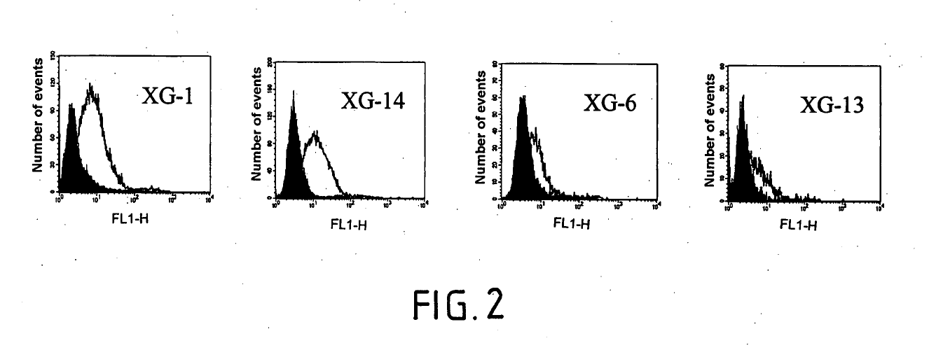

The results obtained are shown in FIG. 2, in which the fluorescence intensity has been plotted on the abscissa and the number of cells counted has been plotted on the ordinate.

These results show that membrane HB-EGF is present on the surface of the cells. The labeling was more intense with the XG-1 and XG-14 cells, which exhibited a stronger expression of the HB-EGF gene, determined by the ‘cytokine/receptor’ DNA chip technique or by RT-PCR, as shown by the data in Table 1 below:

TABLE 1
Gene expression determined by ATLAS DNA membranes (the values
below 20 are considered as non-significant)

| | XG-1 | XG-14 | XG-6 | XG-13 |
|---|---|---|---|---|
| HB-EGF | 2890 | 1020 | 263 | 166 |
| EGF | 6 | 1 | 1 | 54 |
| ErbB1 | 5549 | 3635 | 559 | 783 |
| ErbB2 | 71 | 75 | 60 | 42 |
| ErbB3 | 35 | 52 | 124 | 101 |
| ErbB4 | 17 | 9 | 180 | 25 |

### EXAMPLE 2

### Inhibition of the IL-6-Induced Proliferation of Myeloma Cells by Mutated Diphtheria Toxin

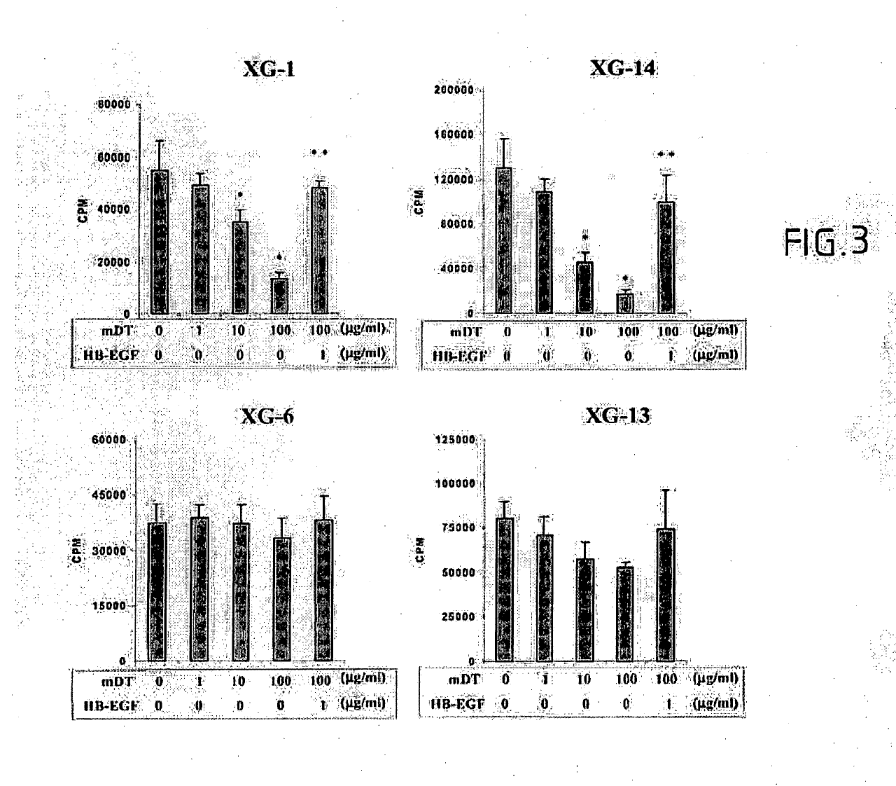

Myeloma cells (10⁴ cells/well) were cultivated for 5 days in X-VIVO 20 serum-free culture medium with 500 μg/ml of IL-6 and a gradually increasing concentration of mutated diphtheria toxin (mDT). In one culture group, 1 μg/ml of recombinant HB-EGF was added at the start of the culture together with 100 μg/ml of mDT and 500 μg/ml of IL-6. The results are the means±SE of the incorporation of tritiated thymidine, determined on six culture wells. The results shown in FIG. 3 are those of one experiment representative of 3 to 4 experiments, according to the cell lines. * indicates a statistical difference in the mean value relative to that of the group of cells cultivated without mDT or HB-EGF (P<0.05, tested by a Student T test). ** indicates a statistical difference in the mean value relative to that of the group of cells cultivated with 100 μg/ml of mDT.

FIG. 3 shows that this autocrine HB-EGF is critical for promoting the growth of 2/4 IL-6-dependent HMCLs, namely HMCLs XG-1 and XG-14. In reality, mutated diphtheria toxin (mDT), which is a specific inhibitor of HB-EGF, caused the IL-6-induced proliferation of HMCLs to decrease. The inhibitory effect of mDT was compensated by the addition of excess recombinant HB-EGF, which indicates that said effect was not due to a non-specific toxicity of mutated DT (FIG. 3).

### EXAMPLE 3

### An HB-EGF Antagonist Does not Inhibit the Proliferation of Myeloma Cells Cultivated With High Concentrations of IL-6

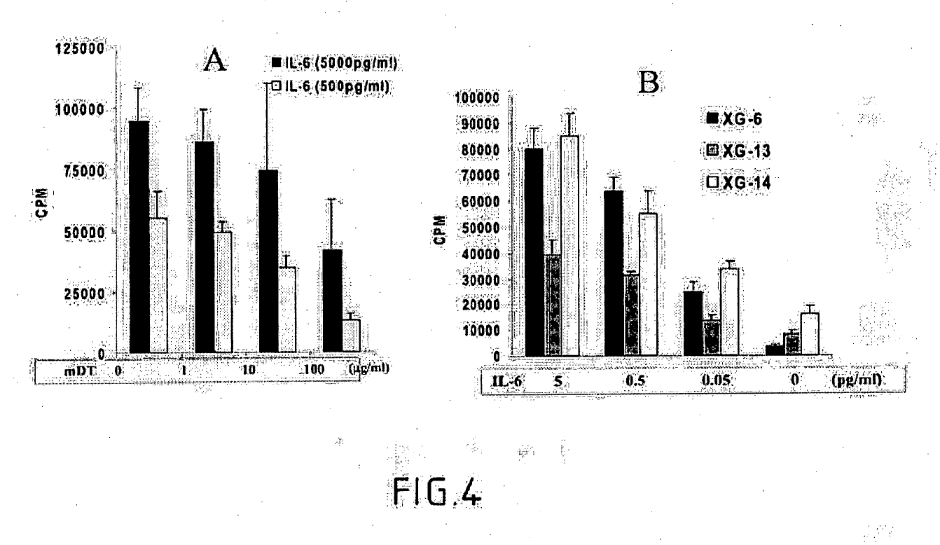

Myeloma cells (10⁴ cells/well) were cultivated for 5 days in X-VIVO 20 serum-free culture medium, either (A) with 500 μg/ml or 5 ng/ml of IL-6 and a gradually increasing concentration of mutated diphtheria toxin (mDT), or (B) with gradually increasing concentrations of IL-6. The results shown in FIG. 4 are means±SE of the incorporation of tritiated thymidine, determined on six culture wells. The results are those of one experiment representative of two experiments.

Inhibition of the IL-6-dependent proliferation of myeloma cells by mDT or anti-HB-EGF antibodies was observed reproducibly when myeloma cells were stimulated with an IL-6 concentration of 100-500 μg/ml (FIG. 4a). With a greater IL-6 concentration (5 ng/ml), no statistically significant inhibition could be observed (FIG. 4a). It should be pointed out that a high degree of proliferation of the 4 HMCLs was already achieved with 100-500 μg/ml of L-6 and could not be increased by the addition of 10-30 times more IL-6 (FIG. 4b).

### EXAMPLE 4

### Induction of the Apoptosis of Myeloma Cells by an HB-EGF Antagonist

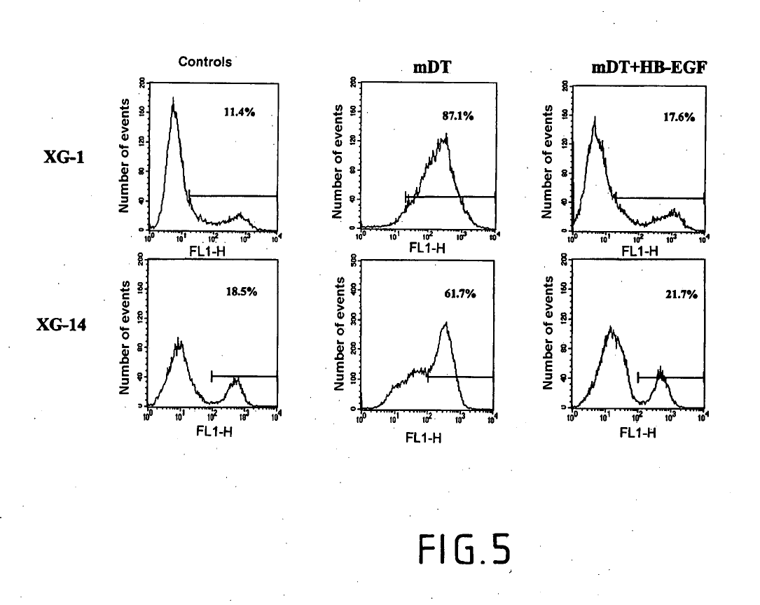

Myeloma cells were cultivated for 3 days with 500 μg/ml of IL-6 and with or without 100 μg/ml of mutated diphtheria toxin. In one group, 1 μg/ml of HB-EGF was added at the start of the culture together with 500 μg/ml of IL-6 and 100 μg/ml of mutated diphtheria toxin. The apoptosis was evaluated by labeling with annexin V and cytofluorimetric analysis. The numbers in the panels indicate the percentage of annexin V-positive cells in apoptosis. The results shown in FIG. 5 are those of one experiment representative of two experiments.

Through labeling with annexin V, mDT was shown to induce apoptosis in the 2 HMCLs XG-1 and XG-14 (FIG. 5), the majority of myeloma cells (87% and 62%) being in apoptosis with 100 μg/ml of mDT. The mDT-induced apoptosis was compensated by the addition of a large amount of recombinant HB-EGF capable of counterbalancing the mDT (FIG. 5).

### EXAMPLE 5

### Expression of ErbB1 in Myeloma Cells

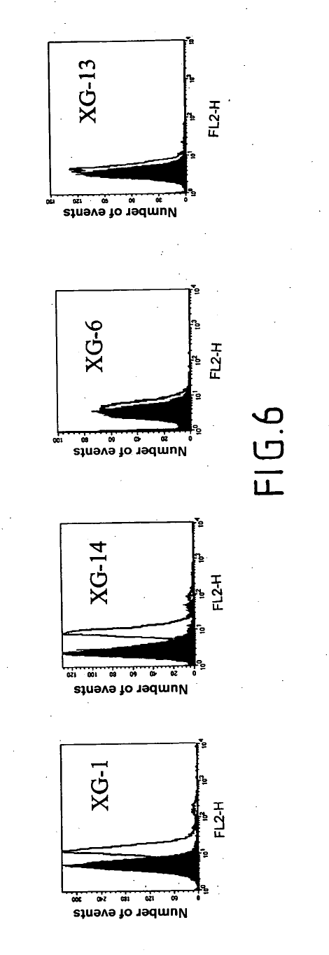

Myeloma cells were labeled with an anti-ErbB1 monoclonal antibody or a control murine monoclonal antibody that does not recognize any human antigens. The cells were then labeled with an anti-murine Ig goat antibody conjugated with PE. The fluorescence was analyzed with a FACScan cytofluorimeter. The results shown in FIG. 6 are those of one experiment representative of three experiments. The XG-1 and XG-14 myeloma cells expressed the greatest density of ErbB1.

These results are consistent with those in Table 1, showing that the myeloma cells express the ErbB1 gene strongly and the other receptors of the EGF-R family more weakly and non-reproducibly.

### EXAMPLE 6

### Inhibition of the L-6-Induced Proliferation of Myeloma Cells by Anti-ErbB1 Monoclonal Antibodies

Myeloma cells (10⁴ cells/well) were cultivated for 5 days in X-VIVO 20 serum-free culture medium with 500 μg/ml of L-6 and a gradually increasing concentration of an anti-ErbB1 monoclonal antibody (0-10 μg/ml). In one culture group, 1 μg/ml of recombinant HB-EGF was added at the start of the culture together with 10 μg/ml of anti-ErbB1 monoclonal antibody and 500 μg/ml of IL-6. The results are means±SE of the incorporation of tritiated thymidine, determined on six culture wells.

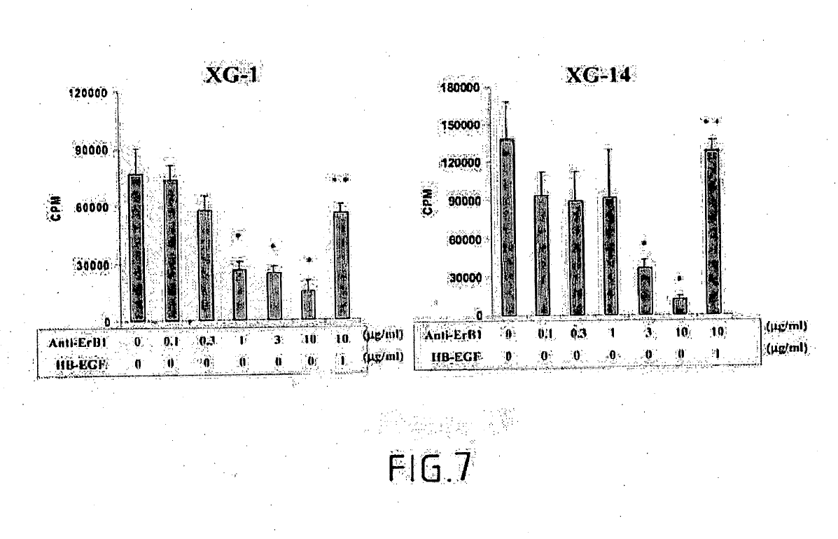

The results shown in FIG. 7 are those of one experiment representative of two to three experiments, according to the cell lines. * indicates a statistical difference in the mean relative to that of the group of cells cultivated without anti-ErbB1 mAb or HB-EGF (p<0.05, tested by a Student T test). ** indicates a statistical difference in the mean relative to that of the group of cells cultivated with 10 μg/ml of anti-ErbB1 mAb.

The results in FIG. 7 show that the proliferation of XG-1 and XG-14 cells was strongly inhibited by the anti-ErbB1 antibody at one concentration (10 μg/ml). The inhibitory effect of the anti-ErbB1 monoclonal antibody was compensated by the addition of a large amount of recombinant HB-EGF. It should be pointed out that myeloma cell lines do not express the EGF gene (Table 1). The strong inhibition of the proliferation of XG-1 and XG-14 cells by the anti-ErbB1 monoclonal antibody is consistent with their high expression of the HB-EGF gene, a marked inhibition by HB-EGF antagonists and an expression of ErbB1 detectable by FACS. Overall, these data show that the IL-6-induced survival and proliferation of XG-1 and XG-14 myeloma cell lines depends on an HB-EGF/ErbB1 autocrine loop.

### EXAMPLE 7

### Inhibition of the L-6-Induced Proliferation of Myeloma Cells by Anti-IL-6 or Anti-ErbB1 Monoclonal Antibodies

XG-1 myeloma cells were cultivated in the presence of 100 μg/ml of interleukin-6 (IL-6) in X-VIVO 20 medium for 96 hours.

On day 0, different concentrations of an anti-IL-6 monoclonal antibody (B-E8) and/or an anti-ErbB1 monoclonal antibody (LA-1) were added.

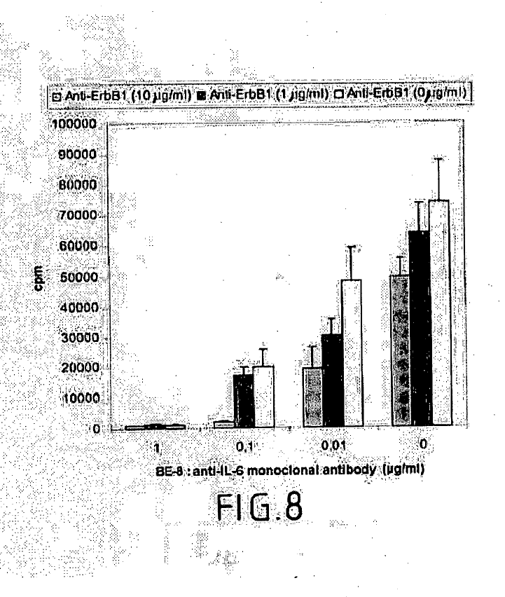

The results in FIG. 8 show that the anti-ErbB1 monoclonal antibody potentiates the inhibitory effect of the anti-IL-6 monoclonal antibody on the IL-6-dependent proliferation of the cells.

### EXAMPLE 8

### Expression of Tetraspanin CD9 by Myeloma Lines

Myeloma cells were cultivated for 2 days in X-VIVO 20 culture medium with 0.2 ng/ml or 2 ng/ml of IL-6, and the expression of CD9 was evaluated by labeling with an anti-CD9 monoclonal antibody conjugated with phycoerythrin. The percentage of labeled cells and the mean fluorescence intensity (MFI) were determined with a FACScan cytofluorimeter. The results are those of one experiment representative of two experiments.

The MFI obtained with the control antibody of corresponding isotype was set between 3 and 5. The results in Table 2 (below) show that the XG-1 and XG-14 lines strongly express tetraspanin CD9. This expression is not regulated by IL-6. The XG-1 and XG-13 lines express it very weakly. As tetraspanin CD9 is an HB-EGF receptor capable of increasing its biological activity very greatly, these data reinforce the importance of a CD9/HB-EGF/ErbB1 autocrine loop in controlling the IL-6-mediated proliferation of the XG-1 and XG-14 lines.

TABLE 2
Expression of CD9 in myeloma cells

| | XG-1 | | XG-14 | | XG-6 | | XG-13 | |
|---|---|---|---|---|---|---|---|---|
| | Labeled | | Labeled | | Labeled | | Labeled | |
| IL-6 | viable cells (%) | MFI | viable cells (%) | MFI | viable cells (%) | MFI | viable cells (%) | MFI |
| 2 ng/ml | 100 | 418 | 100 | 108 | 35 | 17 | 37 | 18 |
| 0.2 ng/ml | 100 | 393 | 100 | 91 | 30 | 19 | 39 | 17 |

### EXAMPLE 9

### Inhibition of the Proliferation of Myeloma Cells by an Anti-CD9 Monoclonal Antibody

An anti-CD9 mAb was used to examine whether CD9 is critical in promoting the L-6-mediated survival of myeloma cells.

Myeloma cells (10⁴ cells/well) were cultivated for 5 days in X-VIVO 20 serum-free culture medium with 500 μg/ml of IL-6 and 50 μg/ml of the anti-CD9 mAb SYB-1. In one culture group, 1 μg/ml of recombinant HB-EGF was added at the start of the culture together with 10 μg/ml of anti-CD9 mAb SYB-1 and 500 μg/ml of IL-6. The results are means±SE of the incorporation of tritiated thymidine, determined on six culture wells. The results are those of one experiment representative of two experiments. * indicates a statistical difference in the mean relative to that of the group of cells cultivated without anti-CD9 mAb or HB-EGF (P<0.05, tested by a Student T test).

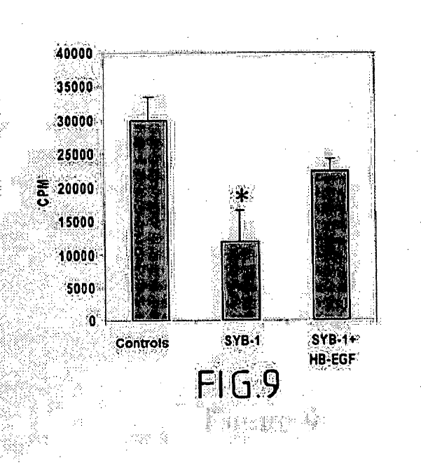

As shown in FIG. 9, the anti-CD9 monoclonal antibody SYB-1 was able to block the proliferation of XG-1 myeloma cells. This inhibition was compensated by the addition of a large amount of recombinant HB-EGF, which is capable of competing with the anti-CD9 monoclonal antibody for binding to CD9.

### EXAMPLE 10

### Synergistic Effects of IL-6 and HB-EGF in Triggering the Survival and Proliferation of Myeloma Cells

XG-1 or XG-14 myeloma cells were cultivated at a rate of 10⁵ cells/ml in X-VIVO 20 serum-free culture medium with 10 μg/ml of a murine monoclonal antibody that does not recognize any human antigens, and without cytokine, or with 500 μg/ml of IL-6 or 100 ng/ml of recombinant HB-EGF. In some culture groups, 10 μg/ml of neutralizing anti-L-6 gp130 transducer monoclonal antibody B-R3 or neutralizing anti-ErbB1 monoclonal antibody LA-I were added. Every 3 to 4 days the viability of the cells and the number of cells were tested, and the cells were cultivated again at a rate of 10⁵ cells/ml with fresh culture medium containing the initial concentrations of cytokine and/or cytokine inhibitor for each group. The results are the cumulative numbers of cells produced in the culture of one experiment representative of three experiments.

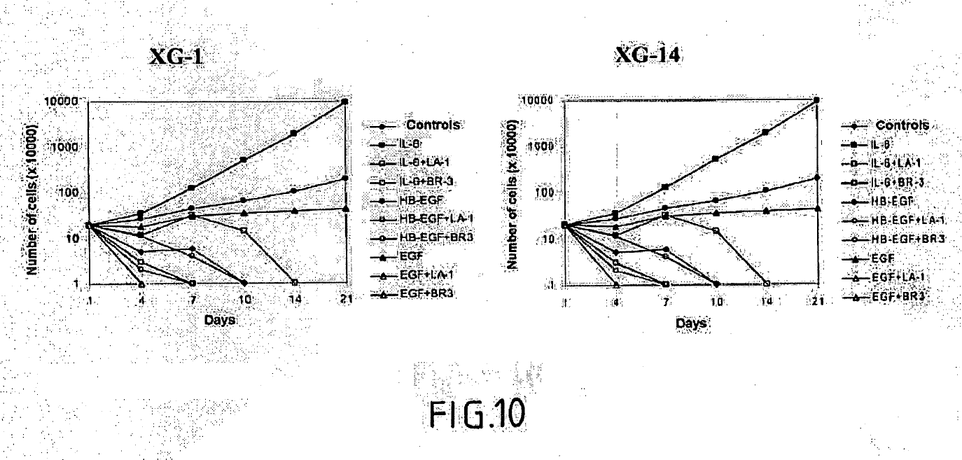

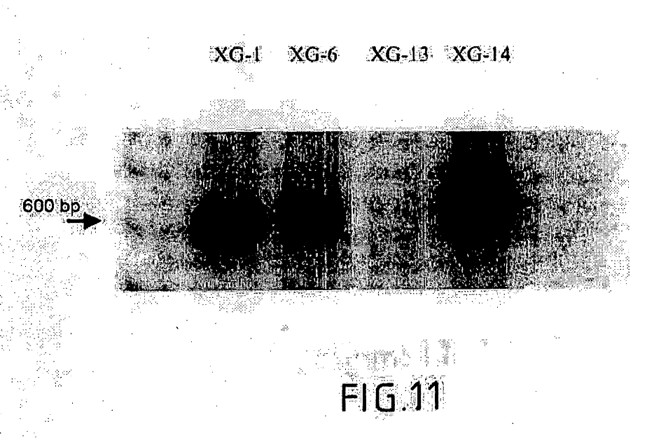

As shown in FIG. 10, in the absence of IL-6 the two myeloma cell lines XG-1 and XG-14 did not develop and gradually died in 4 to 5 days. The addition of IL-6 induced a vigorous growth. The IL-6-induced growth was totally canceled by the neutralizing anti-gp130 mAb. It was also totally canceled by the neutralizing anti-ErbB1 monoclonal antibody, in agreement with the above data. Recombinant HB-EGF favored the survival of XG-1 and XG-14 myeloma cells and a growth which was weaker than that induced by IL-6. The weak growth of the myeloma cells mediated by recombinant HB-EGF was inhibited by the anti-ErbB1 monoclonal antibody. It was also totally inhibited by the neutralizing anti-gp130 monoclonal antibody. This autocrine expression of the IL-6 gene was also detected with the ATLAS DNA chips in XG-1 cells and other myeloma cell lines (cf. Table 1) and confirmed by RT-PCR (FIG. 11).

Taken in combination, these data indicate that the weak growth of myeloma cells with recombinant HB-EGF is linked to this weak autocrine production of IL-6 myeloma cells. From this it is deduced that there is a cooperation between the transduction pathways induced by the IL-6 gp130 transducer and ErbB1 for triggering the optimum survival and proliferation of myeloma cells.

### REFERENCES

1. Kawano M., Hirano T., Matsuda T., Taga T., Horii Y., Iwato K., Asaoka H., Tang B., Tanabe O., Tanaka H., Kuramoto A., Kishimoto T. Autocrine generation and essential requirement of BSF-2/IL-6 for human multiple myeloma.

Nature 1988, 332, 83-85.

2. Klein B., Zhang X. G., Jourdan M., Content J., Houssiau F., Aarden L., Piechaczyk M., Bataille R. Paracrine rather than autocrine regulation of myeloma-cell growth and differentiation by interleukin-6. Blood 1989, 73, 517-526.

3. Portier M., Rajzbaum G., Zhang X. G., Attal M., Rusalen C., Wijdenes J., Mannoni P., Maraninchi D., Piechaczyk M., Bataille R., Klein B. In vivo interleukin-6 gene expression in the tumoral environment in multiple myeloma.

Eur. J. Immunol. 1991, 21, 1759-1762.

4. Chauhan D., Uchiyama H., Akbarali Y., Urashima M., Yamamoto K., Libermann T. A., Anderson K. C. Multiple myeloma cell adhesion-induced interleukin-6 expression in bone marrow stromal cells involves activation of NF-kappa b. Blood 1996, 87, 1104-1112.

5. Lokhorst H. M., Lamme T., de Smet M., Klein S., de Weger R. A., van Oers R., Bloem A. C. Primary tumor cells of myeloma patients induce interleukin-6 secretion in long-term bone marrow cultures. Blood 1994, 84, 2269-2277.

6. Davis-Fleischer K. M., Besner G. E. Structure and function of heparin-binding EGF-like growth factor (HB-EGF). Front Biosci. 1998, 3, d288-299.

7. Iwamoto R., Mekada E. Heparin-binding EGF-like growth factor: a juxtacrine growth factor. Cytokine Growth Factor Rev. 2000, 11, 335-344.

8. Zhang X. G., Gaillard J. P., Robillard N., Lu Z. Y., Gu Z. J., Jourdan M., Boiron J. M., Bataille R., Klein B. Reproducible obtaining of human myeloma cell lines as a model for tumor stem cell study in human multiple myeloma. Blood 1994, 83, 3654-3663.

9. Rebouissou C., Wijdenes J., Autissier P., Tarte K., Costes V., Liautard J., Rossi J. F., Brochier J., Klein B. A gp130 Interleukin-6 Transducer-Dependent SCID Model of Human Multiple Myeloma. Blood 1998, 91, 4727-4737.

10. Gu Z. J., Wijdenes J., Zhang X. G., Hallet M. M., Clement C., Klein B.

Anti-gp130 transducer monoclonal antibodies specifically inhibiting ciliary neurotrophic factor, interleukin-6, interleukin-11, leukemia inhibitory factor or oncostatin M. J. Immunol. Methods 1996, 190, 21-27.

11. Wijdenes J., Clement C., Klein B., Morel-Fourrier B., VIta N., Ferrara P., Peters A. Human recombinant dimeric IL-6 binds to its receptor as detected by anti-IL-6 monoclonal antibodies. Mol. Immunol. 1991, 28, 1183.

12. De Vos J., Jourdan M., Tarte K., Jasmin C., Klein B. JAK2 tyrosine kinase inhibitor tyrphostin AG490 downregulates the mitogen-activated protein kinase (MAPK) and signal transducer and activator of transcription (STAT) pathways and induces apoptosis in myeloma cells. Br. J. Haematol. 2000, 109, 823-828.

13. Lu Z. Y., Brailly H., Widjenes J., Bataille R., Rossi J. F., Klein B.

Measurement of whole body interleukin-6 (IL-6) production: prediction of the efficacy of anti-IL-6 treatments. Blood 1995, 86, 3123-3131.

1-8. (canceled).

9. A method of treating myeloma, comprising administering to a mammal a composition comprising
(a) a pharmaceutically acceptable carrier; and
(b) a pharmaceutically effective amount of a member selected from the group consisting of at least one heparin-binding epidermal growth factor (HB-EGF) inhibitor;
at least one HB-EGF receptor inhibitor or ErbB receptor inhibitor; and
at least one inhibitor of associated transduction pathways.

10. The method of claim 9 wherein the treatment inhibits proliferation of IL-6-dependent plasmocytic tumor cells and/or induces apoptosis of IL-6-dependent plasmocytic tumor cells.

11. The method of claim 9 wherein the HB-EGF inhibitor is a heparin.

12. The method of claim 11, wherein the heparin is selected from the group consisting of low molecular heparin, diphtheria toxin and anti-HB-EGF antibodies.

13. The method of claim 9 wherein the at least one HB-EGF receptor inhibitor is selected from the group consisting of monoclonal antibodies directed against ErbB receptors or other HB-EGF receptors; inhibitors of associated transduction pathways.

14. The method of claim 9 wherein the IL-6 inhibitor is selected from the group consisting of corticoids, inhibitors of IL-6 production, anti-IL-6 monoclonal antibodies and antagonistic mutated interleukin-6.

15. The method of claim 9 wherein the IL-6 receptor inhibitor is directed against gp80 chain or gp130 chain or is an inhibitor of associated transduction pathways.

16. The method of claim 9 wherein the mammal is a human.

17. A method of treating myeloma, comprising administering to a mammal a composition comprising
(a) a pharmaceutically acceptable carrier;
(b) a pharmaceutically effective amount of a member selected from the group consisting of
at least one heparin-binding epidermal growth factor (HB-EGF) inhibitor;
at least one HB-EGF receptor inhibitor or ErbB receptor inhibitor; and
at least one inhibitor of associated transduction pathways; and
(c) a pharmaceutically effective amount of a member selected from the group consisting of
at least one IL-6 inhibitor;
at least one IL-6 receptor inhibitor; and
at least one inhibitor of associated transduction pathways.

18. The method of claim 17 wherein the treatment inhibits proliferation of IL-6-dependent plasmocytic tumor cells and/or induces apoptosis of IL-6-dependent plasmocytic tumor cells.

19. The method of claim 17 wherein the HB-EGF inhibitor is a heparin.

20. The method of claim 19, wherein the heparin is selected from the group consisting of low molecular heparin, diphtheria toxin and anti-HB-EGF antibodies.

21. The method of claim 17 wherein the at least one HB-EGF receptor inhibitor is selected from the group consisting of
monoclonal antibodies directed against ErbB receptors or other HB-EGF receptors; and
inhibitors of associated transduction pathways.

22. The method of claim 17 wherein the IL-6 inhibitor is selected from the group consisting of corticoids, inhibitors of IL-6 production, anti-IL-6 monoclonal antibodies and antagonistic mutated interleukin-6.

23. The method of claim 17 wherein the IL-6 receptor inhibitor is directed against a gp80 or a gp130 chain or is an inhibitor of associated transduction pathways.

24. The method of claim 17 wherein the mammal is a human.

25. A pharmaceutical composition comprising
(a) a pharmaceutically acceptable carrier; and
(b) a pharmaceutically effective amount of a member selected from the group consisting of
at least one heparin-binding epidermal growth factor (HB-EGF) inhibitor;
at least one ErbB receptor inhibitor; and
at least one inhibitor of associated transduction pathways.

26. A pharmaceutical composition comprising
(a) a pharmaceutically acceptable carrier;
(b) a pharmaceutically effective amount of a member selected from the group consisting of
at least one heparin-binding epidermal growth factor (HB-EGF) inhibitor;
at least one HB-EGF receptor inhibitor; and
at least one inhibitor of associated transduction pathways; and
(c) a pharmaceutically effective amount of a member selected from the group consisting of
at least one IL-6 inhibitor;
at least one IL-6 receptor inhibitor; and
at least one inhibitor of associated transduction pathways.
